SEQUENCE LISTING (15 sequences)

SEQ ID NO: 1 - DNA, 903 bp, artificial sequence
  misc_feature (1)..(903): coding sequence of fusion protein
aattccgatagcgagtgtcctctgagtcacgatggttactgtctacatgacggcgtctgtatgtatattgaggctctagacaagtacgcgtgtaattgcgttgttggctacatcggtgagcgctgtcagtatcgagatctgaaatggtgggaacttagaggtggaggcggttcaggcggaggtggctctggcggtggcggatcgagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagt

SEQ ID NO: 2 - protein, 301 aa, artificial sequence
  misc_feature (1)..(301): fusion protein
NSDSECPLSHDGYCLHDGVCMYIEALDKYACNCVVGYIGERCQYRDLKWWELRGGGGSGGGGSGGGGSSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 3 - DNA, 1107 bp, artificial sequence
  misc_feature (1)..(1107): coding sequence of fusion protein
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaaatgtgacaagccgaggcggggtggaggcggttcaggcggaggtggctctggcggtggcggatcgagcgagaaaagcgaagaaataaatgaaaaagatttgcgaaaaaagtctgaattgcagggaacagctttaggcaatcttaaacaaatctattattacaatgaaaaagctaaaactgaaaataaagagagtcacgatcaatttttacagcatactatattgtttaaaggcttttttacagatcattcgtggtataacgatttattagtagattttgattcaaaggatattgttgataaatataaagggaaaaaagtagacttgtatggtgcttattatggttatcaatgtgcgggtggtacaccaaacaaaacagcttgtatgtatggtggtgtaacgttacatgataataatcgattgaccgaagagaaaaaagtgccgatcaatttatggctagacggtaaacaaaatacagtacctttggaaacggttaaaacgaataagaaaaatgtaactgttcaggagttggatcttcaagcaagacgttatttacaggaaaaatataatttatataactctgatgtttttgatgggaaggttcagaggggattaatcgtgtttcatacttctacagaaccttcggttaattacgatttatttggtgctcaaggacagtattcaaatacactattaagaatatatagagataataaaacgattaactctgaaaacatgcatattgatatatatttatatacaagt

SEQ ID NO: 4 - protein, 369 aa, artificial sequence
  misc_feature (1)..(369): fusion protein
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRRGGGGSGGGGSGGGGSSEKSEEINEKDLRKKSELQGTALGNLKQIYYYNEKAKTENKESHDQFLQHTILFKGFFTDHSWYNDLLVDFDSKDIVDKYKGKKVDLYGAYYGYQCAGGTPNKTACMYGGVTLHDNNRLTEEKKVPINLWLDGKQNTVPLETVKTNKKNVTVQELDLQARRYLQEKYNLYNSDVFDGKVQRGLIVFHTSTEPSVNYDLFGAQGQYSNTLLRIYRDNKTINSENMHIDIYLYTS

SEQ ID NO: 5 - DNA, 45 bp, artificial sequence
  misc_feature (1)..(45): primer
ggtggaggcggttcaggcggaggtggctctggcggtggcggatcg

SEQ ID NO: 6 - protein, 15 aa, artificial sequence
  misc_feature (1)..(15): linker peptide
GGGGSGGGGSGGGGS

SEQ ID NO: 7 - DNA, 34 bp, artificial sequence
  misc_feature (1)..(34): primer
gagcccgggcagcgagaaaagcgaagaaataaat

SEQ ID NO: 8 - DNA, 40 bp, artificial sequence
  misc_feature (1)..(40): primer
gtgcggccgcacttgtatataaatatatatcaatatgcat

SEQ ID NO: 9 - DNA, 28 bp, artificial sequence
  misc_feature (1)..(28): primer
gagcccgggcaattccgatagcgagtgt

SEQ ID NO: 10 - DNA, 28 bp, artificial sequence
  misc_feature (1)..(28): primer
gtgcggccgctctaagttcccaccattt

SEQ ID NO: 11 - DNA, 31 bp, artificial sequence
  misc_feature (1)..(31): primer
gagcccgggcgcacccatggcagaaggagga

SEQ ID NO: 12 - DNA, 55 bp, artificial sequence
  misc_feature (1)..(55): primer
gtgcggccgcccgcctcggcttgtcacatttttcttgtcttgctctatctttctt

SEQ ID NO: 13 - DNA, 54 bp, artificial sequence
  misc_feature (1)..(54): primer
gccagagccacctccgcctgaaccgcctccacctctaagttcccaccatttcag

SEQ ID NO: 14 - DNA, 60 bp, artificial sequence
  misc_feature (1)..(60): primer
tcaggcggaggtggctctggcggtggcggatcgagcgagaaaagcgaagaaataaatgaa

SEQ ID NO: 15 - DNA, 57 bp, artificial sequence
  misc_feature (1)..(57): primer
gccagagccacctccgcctgaaccgcctccaccccgcctcggcttgtcacatttttc

(19) United States
(12) Patent Application Publication
(10) Pub. No.: US 2007/0092528 A1
(43) Pub. Date: Apr. 26, 2007

(54) Superantigen fusion protein for anti-cancer therapy and methods for the production thereof

(76) Inventor: Jialin Sun, Shanghai (CN)

Correspondence Address: HAMRE, SCHUMANN, MUELLER & LARSON, P.C., P.O. BOX 2902, MINNEAPOLIS, MN, 55402-0902, US

(21) Appl. No.: 10/571,836

(22) PCT Filed: May 31, 2004
(86) PCT No.: PCT/CN04/00569

§ 371 Date: Mar. 15, 2006

(30) Foreign Application Priority Data
Dec. 21, 2003 (CN) 20031019829.7

(51) Int. Cl. A61K 39/00; C07H 21/04; C12P 21/04; C07K 14/475

(52) U.S. Cl. 424/185.1; 435/69.7; 435/320.1; 435/325; 530/350; 536/23.5

(57) ABSTRACT

The present invention provides a fusion protein, comprising: a) a ligand that stimulates cancer cell growth and corresponds to receptors overexpressed by cancer cells, or a screened peptide that is affinitive to or antagonist to cancer cell receptors, or a peptide that directly interacts with cancer cell surface; b) a superantigen that may lead to anti-cancer immune response. It also discloses expression vectors and host cells comprising this fusion protein, methods for preparing this fusion protein, and the application of this fusion protein to prepare therapeutic agents for cancer or immune disease treatment.

### FIELD OF THE INVENTION

The invention relates to molecular biology field, in particular, to a fusion protein. It also discloses expression vectors and host cells comprising this fusion protein, and methods for the preparation thereof.

### BACKGROUND

At present, drug therapy for cancer has mainly involved the use of chemotherapeutic agents, which have severe side effects. Chemotherapeutic agents not only kill cancer cells, but also damage normal cells. Chemotherapeutic agents are lack of cancer specificity.

Antibodies offer an excellent opportunity for solving the problem of drug specificity. They're commonly used specific cancer-cell-targeting carriers, which may specifically affect cancer cells. Antibodies themselves can block cancer cells, their Fc fragments may lead to cytotoxicity. Antibodies can also be conjugated to a toxin protein, and direct the toxin protein to kill cancer cells

Superantigens can also lead to cytotoxicity. They are a class of special antigens, mainly include certain bacterial toxins and retrovirus gene products. Without being processed by antigen presenting cells (APC), superatnigens, as intact proteins, directly bind to and form complex with MHC Class II molecules on the cellular membrane. They recognize the T cell receptor (TCR) V β chain and activate much more T lymphocytes (including CD4⁺, CD8⁺) than conventional antigens do. Furthermore, superantigens also induce the release of a large amount of cytokines and produce effective cytotoxicity on targeted cells.

Superantigens are responsible for a number of human acute/chronic diseases, and also play a distinctive role in anti-tumor research. The attempt to kill tumors by superantigen-activated T lymphocytes has already shown encouraging results. The presently well-studied superantigens are Staphylococcus aureus enterotoxin A and B. Since superantigens are lacking of anti-tumor specificity, they may also effect on normal cells expressing MHC II molecules. Therefore, the clinical application of superantigens for anti-cancer therapy is predicted to be largely limited by side effects.

To solving the problem of lacking anti-tumor specificity of superantigens, they are fused to antibodies, and the anti-tumor antibodies direct superantigen Staphylococcal enterotoxin A (SEA) to cancer cells (M. Dohlsten, et al, Proc. Natl. Acad. Sci. USA, 91, 8945-8949, 1994; J. Ihle, et al, Cancer Res., 55, 623-628, 1995). The SEA gene was reported in the 1980s (I. Y. Huang, et al, J. Biol. Chem., 262, 7006-7013, 1987; M. J. Betley and J. J. Mekalanos, J. Bacteriol., 170, 34-41, 1988).

To make antibodies into medicaments, murine antibodies need to be humanized by genetic engineering techniques. Since the dosage of antibodies is quite high, usually tens of mg/person/time, it requests to increase the expression level of genetic engineering antibodies in animal cells and develop large-scale fermentation techniques. Therefore, the period of research and development (R&D) for therapeutic antibody agents is extremely long and the cost of investment is considerably huge.

Aside from antibodies, cytokines associated with cancer cell growth are also used to direct cancer cell specificity. For example, epidermal growth factor (EGF) is linked to RNase (H. Jinno, et al, Cancer Chemother. Pharmacol., 38, 303-308, 1996) and toxin (A. Schmidt, et al, Biochem. Biophys. Res. Commun., 277, 499-506, 2000); basic fibroblast growth factor (bFGF), vascular endothelial cell growth factor (VEGF) and transforming growth factor-α (TGF-α) are also used to construct fusion proteins with toxin, respectively (Biochem. Biophys. Res. Commun., 277, 499-506, 2000; L. M. Veenendaal, et al, Proc. Natl. Acad. Sci. USA, 99, 7866-7871, 2002; A. Kihara and I. Pastan, Cancer Res., 54, 5154-5159, 1994). Other cytokines have also been reported, for example, interleukin-4 (IL-4) and interleukin-2 (IL-2) are fused to toxin, respectively (S. R. Husain, et al, Cancer Res., 58, 3649-3653, 1998; J. M. Dore, et al, FEBS Lett., 402, 50-52, 1997).

EGF gene was found in the early 1980s (J. Smith, et al, Nucleic Acids Res., 10, 4467-4482, 1982; A. Gray, et al, Nature, 303, 722-725, 1983), mature EGF is a polypeptide consisting of 53 amino acids.

VEGF gene was found in the late 1980s (D. W. Leung, et al, Science, 246, 1306-1309, 1989; P. J. Keck, et al, Science, 246, 1309-1312, 1989). According to differential mRNA splicing, mature VEGF has several isoforms, ranging from 189, 165 and 121 amino acids in length (E. Tischer, et al, J. Biol. Chem., 266, 11947-11954, 1991).

All the works above are based on the same strategy, that is, to construct fusion proteins comprising a cytokine fused to a protein toxin or RNase (E. B. Sweeney and J. R. Murphy, Essays Biochem., 30, 119-131, 1995). The presence of these cytokines allows specific directing to cancer cells, then the protein toxin and RNase operate cancer-cell-specific killing. However, the action mechanism is different from those of antibody Fc fragment and superantigen, the latter two mobilize immune system to stimulate anti-cancer cytotoxicity.

Cancer cells are transformed from normal cells, whose antigens are autoantigens. Thus, cancer cells can evade immune surveillance. Attempts have been made over the past years to look for a new anti-cancer method that potentiates the immunity in patients with cancer, especially the specific immunity against cancer cells. Therefore, there is a need in the art for novel effective anti-cancer method specifically targeting cancer cells.

### SUMMARY OF THE INVENTION

Therefore, one purpose of the present invention is to provide a method of specific and effective tumoricidal treatment for cancer.

In one aspect of the present invention, it provides a fusion protein, comprising:

a) a ligand that stimulates cancer cell growth and corresponds to receptors overexpressed by cancer cells, or a screened polypeptide that is affinitive to and antagonistic to cancer cell receptors, or a peptide that directly interacts with cancer cell surface;

b) a superantigen that may lead to anti-cancer immune response.

In a preferred example of this aspect, ligand is selected from epidermal growth factor (EGF) family, vascular endothelial cell growth factor (VEGF) family, basic fibroblast growth factor bFGF and FGF family, transforming growth factor-α (TGF-α), interleukin-2 (IL-2), interleukin-3 (IL-3), interleukin-4 (IL-4), interleukin-6 (IL-6), interleukin-13 (IL-13), granulocyte-macrophage colony-stimulating factor (GM-CSF), heparin-binding EGF-like growth factor (HB-EGF), insulin-like growth factor (IGF), hepatocyte growth factor (HGF), platelet-derived growth factor (PDGF), nerve growth factor (NGF), placental growth factor (PGF), stem cell factor (SCF), interleukin-8 (IL-8), Heregulin, erbB ligand, chemokines, angiopoietin, thrombopoietin, Factor VII, urokinase-type plasminogen activator, growth hormone releasing hormone, somatostatin, asialoglycoprotein, low density lipoprotein, transferrin and other ligands associated with cancer or immune diseases, or their natural and artificial variants with at least 70% identity in amino acid sequence. More preferably, it is selected from epidermal growth factor (EGF) and vascular endothelial cell growth factor (VEGF).

In another preferred example of this aspect, superantigen is selected from Staphylococcal aureus enterotoxin (SE) family, such as SEA, SEB, SEC, SED, SEC and SEE, Streptococcus exotoxin (SPE), such as SPE-A, SPE-B and SPE-C, Staphylococcus aureus toxic shock-syndrome toxin (TSST), Streptococcal mitogenic exotoxin (SME), Streptococcal superantigen (SSA), viral protein and their natural and artificial variants with at least 70% identity in amino acid sequence. More preferably, it is selected from SEA of Staphylococcal enterotoxin (SE) family.

In a preferred example of this aspect, superantigen is SEA protein; ligand is selected from epidermal growth factor (EGF) and vascular endothelial cell growth factor (VEGF).

In a preferred example of this aspect, fusion protein comprises (a) a superantigen; (b) a ligand; (c) a controllable linker to conjugate superantigen and ligand. More preferably, superantigen is SEA protein; ligand is selected from EGF or VEGF; said linker consists of the nucleotide sequence of SEQ ID NO:5. More preferably, the linker encodes the amino acid sequence of SEQ ID NO:6.

In a preferred example of this aspect, fusion protein consists of the amino acid sequence encoded by the nucleotide sequence of SEQ ID NO: 1 or 3. Preferred fusion protein consists of the amino acid sequence of SEQ ID NO: 2 or 4.

In another aspect of the present invention, it provides a recombinant vector, which comprises the nucleotide sequence encoding the fusion protein described above.

In an aspect of the present invention, it provides a host cell, which comprises the recombinant vector described above.

In another aspect of the present invention, it provides a method for producing the fusion protein described above, comprising the steps of: culturing the host cell hereinabove, collecting the expressed fusion protein hereinabove.

In a preferred example of this aspect, it also includes the step of purifying the collected fusion protein.

In an aspect of the present invention, it provides the use of the fusion protein hereinabove in preparation of medicant for treating cancer or immune disease treatment.

### DESCRIPTION OF THE FIGURES

FIG. 1 shows the schematic representation of EGF-SEA fusion protein gene construction by PCR. At first, DNA polynucleotide fragments of EGF and SEA gene were obtained by the first PCR reaction, respectively. Then, the two fragments were ligated by overlap extension PCR, the resulting EGF-SEA fusion protein gene fragment was inserted into an E. coli expressing vector to produce the fusion protein.

FIG. 2 shows the schematic representation of VEGF-SEA fusion protein gene construction by PCR. At first, DNA fragments of VEGF and SEA gene were obtained by the first PCR reaction, respectively. Then, the two fragments were ligated by overlap extension PCR, the resulting VEGF-SEA fusion protein gene fragment was inserted into an E. coli expressing vector to produce the fusion protein.

FIG. 3 shows the SDS-PAGE results of purified EGF-SEA fusion protein.

FIG. 4 shows the SDS-PAGE results of purified VEGF-SEA fusion protein.

FIG. 5 shows the results of tumor cell inhibition assay of EGF-SEA and VEGF-SEA fusion proteins.

### DETAILED DESCRIPTION OF THE INVENTION

To develop specific therapeutic agents against cancer, the present invention constructs a novel cytokine-superantigen fusion protein on the basis of individual characters of superantigen and cytokine. Cytokine, which stimulates cancer cell growth, is capable of directing the fusion protein to cancer cells; while superantigen leads to anti-cancer immune response around cancer cells, i.e. superantigen-dependent cellular cytotoxicity (SDCC). In this manner, this kind of fusion protein may specifically target cancer cells and lead to anti-cancer cytotoxic immune response around cancer cells.

The present invention chooses a new strategy, that is to construct a novel cytokine-superantigen fusion protein by fusing superantigen to a cytokine. As a model, in the present invention, epidermal growth factor (EGF) and vascular endothelial cell growth factor (VEGF) are used to construct the novel fusion protein with superantigen SEA, respectively.

Although herein only superantigen SEA is chosen, superantigen SEB, SEC and other superantigens can also demonstrate the idea of the present invention. The role of superantigen SEA or other superantigens is to activate immune response.

Similarly, the experimental materials epidermal growth factor (EGF) and vascular endothelial cell growth factor (VEGF) are only used to target cancer cells, other cytokines closely associated with cancer cells may also demonstrate the idea of the present invention.

Since fusion protein of the present invention may be constructed by superantigen and various cytokines, a general protein purification method is employed, that is to purify various fusion proteins in the same way. A cellulose binding protein (CBD) is used as a purification Tag in the present method, which is contained in plasmid pET-34b (Novagen Inc.).

Because cancer cells express receptors for these cytokines in large amount, cytokines closely associated with cancer are employed as cancer-cell-targeting carriers. For example, EGF receptors are generally overexpressed on the cancer cell membrane. EGF stimulates cancer cell growth by interacting with EGF receptors. Likewise, cancer tissues receive VEGF signaling via abnormally overexpressing VEGF receptors, which promotes abnormal angiogenesis in cancer tissues and enables the continuous expansion of the entire cancer tissues.

While the expression level of these receptors is undetectable or considerably low on normal cell membrane, cytokines are capable of specific cancer cell targeting. Taking advantage of the cancer-cell-recognizing capacity of EGF and VEGF, superantigen SEA is ligated to EGF and VEGF respectively. This makes it possible to concentrate SEA around cancer cells, and specifically activate immune response and produce formidable cytotoxicity against cancer cells. Single administration of superantigen SEA may result in side effects of systemic administration, while administration of fusion protein concentrates a large amount of SEA-induced cytotoxic T killer cells narrowly around cancer tissues.

Superantigen SEA is related to T lymphocyte activating proliferation and cytotoxicity in a dose-dependent manner, within the range of 0.1 μg˜100 μg per mouse, the maximum effect appears at 24 hours after injection and disappears in 96 hours. The maximum effective concentration of SDCC is 1 μg per mouse, the effective peak appears at 48 hours and disappears in 96 hours (G. Hedlund, et al, Cancer Immunol. Immunother., 36, 89-93, 1993).

Therefore, fusion protein exerts the function similar to antibody-SEA, while this method economizes the cost of drug development, such as humanization of murine antibodies and large-scale animal cell expression. Therefore, therapeutic superantigen SEA agents significantly cut down the production cost of drugs and the medical cost of patients. Likewise, fusion protein of the present invention, comprising superantigen SEA, may significantly decrease the administration dosage.

EGF-SEA and VEGF-SEA only serve as materials to explain the present invention, the idea of the present invention may be expanded. For example, the structure of fusion proteins may be modified by using various cytokines, superantigens and variants thereof, these variants may ameliorate their biological functions and decrease the possible side effects.

The fusion protein may be either in form of EGF-SEA and VEGF-SEA, or in form of SEA-EGF and SEA-VEGF. The two proteins are spatially independent. Therefore, cytokine and superantigen independently function in both forms.

The amino acid composition and length of the linker connecting the two proteins may be in various forms. A too short linker may result in spatial obstruction due to the excessive close connection between cytokine and superantigen. An appropriate linker is essential to the full functions of the cytokine and superantigen.

The fusion protein genes hereinabove may be introduced into recombinant engineered host cells including animal cell, insect cell, plant cell, yeast and bacteria. Fusion proteins may be expressed in various forms, either secreted or unsecreted. Cell-free in vitro translation system may also be employed to produce the fusion proteins.

Fusion proteins can also be constructed by chemical methods, for example, crosslinking, by linking cytokine and superantigen polypeptide fragments, for example, covalent linkage.

Fusion proteins may be put into further modification, such as chemical modification, truncating partial peptide fragment of fusion protein and ligating other peptides to these proteins.

In order to increase their biological activity, purified fusion protein may be consummated by series of protein denaturation and renaturation, which can ameliorate the protein structure including disulfide bond.

The present invention demonstrates a new anti-cancer method, that is, to construct a cytokine-superantigen fusion protein, in which cytokine directs superantigen to cancer cells and results in anti-cancer cytotoxic immune response around cancer cells.

In a greater view, the relation between cytokines and their receptors overexpressed on cancer cell surface is a kind of ligand-receptor interaction in fact. The ligand-receptor affinity may direct superantigen to tumor tissues. Aside from cytokines, other polypeptides, i.e. ligands corresponding to receptors overexpressed by cancer cells may also be used to direct cancer cell specificity. These substances consist of various kinds of chemokines, Ephrin family, angiopoietin (Ang), thrombopoietin (TPO), factor VII, urokinase-type plasminogen activator (uPA), gastrin-releasing peptide (GRP), growth hormone releasing hormone (GHRH), gonadotropin-releasing hormone (GRH), α-melanocyte stimulating hormone (α-MSH), prolactin (PRL), prolactin releasing hormone (PRLH), growth hormone (GH), follicle stimulating hormone (FSH), placental lactogen (PL), chorionic gonadotropin (CG), corticotrophin releasing hormone (CRH), somatostatin (SST), asialoglycoprotein (ASGP), low density lipoprotein (LDL) and transferring (Tf). Many tumor tissues overexpress receptors for these substances. Thus, like cytokines, polypeptide ligands (such as chemokine, enzyme, hormone and other proteins) may be linked to superantigens and form fusion proteins to direct superantigens to tumor tissues.

Aside from ligands corresponding to receptors on cancer cells described above, polypeptides artificially screened (obtained by phage display, etc.) that are affinitive to or antagonist to cancer cell receptors, and other peptides that directly interact with cancer cell surface can also be used to construct fusion protein with superantigens.

Fusion protein exerts specific anti-cancer function similar to antibody, the tumoricidal effect of superantigen-activated T lymphocytes is stronger than that of antibody, while the administration dosage of fusion protein is far lower than that of therapeutic antibody agent. Therefore, the production cost may be significantly cut down.

Pharmaceutical preparations containing the fusion proteins hereinabove may be used in clinical area of anti-cancer and immune diseases. They are formulated with preservatives, emulsions, liposomes, dispersants and stabilizers into agents for injection, oral administration, percutaneous absorption and surgical administration.

Aside from fusion protein itself, nucleotide fragmentss or vectors encoding fusion protein may also be applied in gene therapy. For instance, these nucleotide fragments-are injected into animals and are transfected into cells to express the fusion protein.

A series of primers are designed on the basis of known SEA, EGF and VEGF gene sequences, and these genes are isolated by polymerase chain reaction (PCR). Then, EGF and VEGF genes are ligated with linker and SEA to construct a DNA fragment of fusion protein by PCR, respectively. The resulting gene fragment is inserted into an E. coli. expression vector, fusion proteins are strongly expressed under the control of T7 promoter. Finally, the expressed fusion proteins are isolated and purified.

The encoded protein and peptide forms used in experiments of the present invention are:

(1) mature SEA; (2) EGF of 53 amino acids; (3) VEGF of 121 amino acids; (4) a linker to ligate cytokine and superantigen, whose nucleotide sequence is GGTGGA GGCGGTTCAGGCGGAGGTGGCTCTGGCGGTGGCGGATCG (SEQ ID NO:5) encoding a polypeptide of 15 amino acids GlyGlyGlyGlySerGlyGlyGlyGlySerGly GlyGlyGlySer (SEQ ID NO: ID NO:6).

The chosen E. coli plasmid is pET-34b (Novagen Inc.), about 6 kb in length. It contains a start codon ATG, a stop codon TAA, multiple restriction sites between them, and a CBD-Tag for purification. Here, SrfI and NotI restriction sites are chosen for cloning, kanamycin serves as a selective marker, gene expression is under the control of T7 promoter.

The following examples serve to illustrate the present invention and are not be construed as limiting the scope thereof.

### EXAMPLE 1

### Isolation of Superantigen SEA Gene

According to conventional experimental methods of molecular biology (T. Maniatis, et al, Molecular cloning, A laboratory manual, Second edition, Cold spring harbor laboratory, 1989), DNA was prepared from Staphylococcus aureus FRI337 by phenol/chloroform extraction. Primers were designed on the basis of published superantigen SEA gene sequence (M. J. Betley and J. J. Mekalanos, J. Bacteriol., 170, 34-41, 1988):(1) forward primer containing a SrfI restrition site, 5′-GAGCCCGGGCAGCGAGAAAAGCGAAGAAATAAA T-3′(SEQ ID NO: 7); (2) reverse primer containing a NotI restriction site, 5′-GTGCGGCCGCACTT GTATATAAATATATATCAATATGCAT-3′ (SEQ ID NO: 8). The primers were used for PCR amplification of SEA gene. PCR reaction was performed with 0.1 μl template by 30 cycles of: [95° C. for 30 sec, 55° C. for 30 sec, 72° C. for 120 sec], and finished by 10 min at 72° C. The obtained DNA fragment was about 700 bp in length.

After low melting point agarose gel electrophoresis, the DNA product was extracted and further digested with restriction enzyme SrfI and NotI. The resulting gene fragment was then inserted into pET-34b plasmid. DNA ligation reaction was carried out at 16° C. for 12 hours with DNA ligase. The sequence was confirmed by DNA sequencing at last.

### EXAMPLE 2

### Isolation of Epidermal Growth Factor (EGF) Gene

Primers were designed on the basis of previously reported epidermal growth factor (EGF) gene sequence (J. Smith, et al, Nucleic Acids Res., 10, 4467-4482, 1982): (1) forward primer containing a SrfI restriction site, 5′-GAGCCCGGGCAA TTCCGATAGCGAGTGT-3′(SEQ ID NO:9); (2) reverse primer containing a NotI restriction site, 5′-GTGCGGCCGCTCTAAGTTCCCACCATTT-3′(SEQ ID NO: 10). EGF gene is isolated from human breast cancer cDNA gene library( Clontech Inc.) by PCR, which encodes a polypeptide of 53 amino acids. PCR reaction was performed with 0.1 μl template by 30 cycles of: [95° C. for 30 sec, 55° C. for 30 sec, 72° C. for 30 sec], and finished by 10 min at 72° C. The obtained DNA fragment was about 170 bp in length.

After low melting point agarose gel electrophoresis, the DNA product was extracted and further digested with restriction enzyme SrfI and NotI. The resulting gene fragment was then inserted into pET-34b plasmid. DNA ligation reaction was carried out at 16° C. for 12 hours with DNA ligase. The sequence was confirmed by DNA sequencing at last.

### EXAMPLE 3

### Isolation of Vascular Endothelial Cell Growth Factor (VEGF)

Primers for VEGF-121 were designed on the basis of previously reported vascular endothelial cell growth factor (VEGF) gene sequence (P. J. Keck, et al, Science, 246, 1309-1312, 1989; E. Tischer, et al, J. Biol. Chem., 266, 11947-11954, 1991):(1) forward primer containing a SrfI restriction site, 5′-GAGCCCGGGCGCACCCATGGCAGAAGGAGGA-3′ (SEQ ID NO: 1); (2) reverse primer containing a NotI restriction site, 5′-GTGCGGCCGCCCGCC TCGGCTTGTCACATTTTTCTTGTCTTGCTCTATCTTTCTT-3′ (SEQ ID NO: 12). VEGF-121 gene was isolated from human breast cancer cDNA gene library (Clontech Inc.) by PCR, it encodes a polypeptide of 121 amino acids. PCR reaction was performed with 0.1 μl template by 30 cycles of: [95° C. for 30 sec, 55° C. for 30 sec, 72° C. for 50 sec], and finished by 10 min at 72° C. The obtained DNA fragment was about 370 bp in length.

After low melting point agarose gel electrophoresis, the DNA product was extracted and further digested with restriction enzyme SrfI and NotI. The resulting gene fragment was then inserted into pET-34b plasmid. DNA ligation reaction was carried out at 16° C. for 12 hours with DNA ligase. The sequence was confirmed by DNA sequencing at last.

### EXAMPLE 4

### Construction of EGF-SEA Fusion Protein Gene with Primers Containing Linker Sequence

By overlap extension PCR, EGF and SEA were ligated by a polynucletotide fragment of SEQ ID NO: 6, encoding a polypeptide of 15 amino acids ( GlyGlyGlyGlySerGlyGlyGlyGlySerGlyGlyGlyGlySer) (R. M. Horton, et al, Methods Enzymol., 217, 270-279, 1993)

First pair of primers:

1. forward primer for EGF gene containing a SrfI restriction site, 5′-GAGCCCGGGCAATTCCGATAGCGAGTGT-3′(SEQ ID NO:9);

2. reverse primer for EGF gene containing partial linker, 5′-GCCAGAGCCACCTCCGCCTGAACCGCCTCCACC-TCTAAGTTCCCACCATT TCAG-3′ (SEQ ID NO:13), sequence of linker was underlined.

Second pair of primers:

1. forward primer for mature SEA gene containing partial linker, 5′-TCAGGCGGAGGTGGCTCTGGCGGTGGCGGATCG-AGCGAGAAAAGCGAA GAAATAAATGAA-3′(SEQ ID NO:14), sequence of linker was underlined;

2. reverse primer for SEA gene containing a NotI restriction site, 5′-GTGCGGCCGCACTTGTATATAAATATATATCAATATGCAT-3′(SEQ ID NO: 8).

First, DNA fragments of EGF gene and SEA gene were synthesized using the first pair and second pair of primers respectively, the sequenced genes obtained in Example 1 and Example 2 served as templates. This was the first PCR reaction. After electrophoresis, the gel splice containing desired DNA fragment was cut out. In this way, PCR primers were removed from the DNA product.

Trace amounts of the purified DNA extract of the two gene fragments hereinabove were mixed. After adding DNA polymerase to the mixture, the two DNA fragments were ligated. PCR reaction was performed by 3 cycle of: [95° C. for 30 sec, 55° C. for 30 sec, 72° C. for 150 sec]. The resulting fragment was the template for an additional PCR reaction.

Primer (1) of the first pair and primer (2) of the second pair were re-added for the final PCR reaction. PCR reaction was performed by 30 cycles of: [95° C. for 30 sec, 55° C. for 30 sec, 72° C. for 150 sec], and finished by 10 min at 72° C.

Then, the gene fragment of EGF-SEA fusion protein was constructed.

### EXAMPLE 5

### Construction of VEGF-SEA Fusion Protein Gene with Primers Containing Linker Sequence

As shown in Example 4, overlap extension PCR method was employed.

First pair of primers:

1. forward primer for VEGF gene containing a SrfI restriction site 5′-GAGCCCGGGC GCACCCATGGCAGAAGGAGGA-3′(SEQ ID NO: 11);

2. reverse primer for VEGF gene containing partial linker, 5′-GCCAGAGCCACCTCCGCCTGAACCGCCTCCACC-CCGCCTCGGCTTGTCAC ATTTTTC-3′ (SEQ ID NO: 15), sequence of linker was underlined.

Second pair of primers:

1. forward primer for mature SEA gene containing partial linker, 5′-TCAGGCGGAGGTGGCTCTGGCGGTGGCGGATCG-AGCGAGAAAAGCGAA GAAATAAATGAA-3′(SEQ ID NO:14), sequence of linker was underlined;

2. reverse primer for SEA gene containing a Not I restriction site, 5 ′-GTGCGGCCGCACTTGTATATAAATATATATCAATATGCAT-3 ′(SEQ ID NO:8).

First, DNA fragments of VEGF gene and SEA gene were synthesized using the first pair and second pair of primers respectively, the sequenced genes obtained in Example 3 and Example 1 served as templates. This was the first PCR reaction. After electrophoresis, the gel splice containing desired DNA fragment was cut out. In this way, PCR primers were removed from the DNA product.

Trace amounts of the purified DNA extract of the two gene fragments hereinabove were mixed. After adding DNA polymerase to the mixture, the two DNA fragments were ligated. PCR reaction was performed by 3 cycle of: [95° C. for 30 sec, 55° C. for 30 sec, 72° C. for 150 sec]. The resulting fragment was the template for an additional PCR reaction.

Primer (1) of the first pair and primer (2) of the second pair were re-added for the final PCR reaction. PCR reaction was performed by 30 cycles of: [95° C. for 30 sec, 55° C. for 30 sec, 72° C. for 150 sec], and finished by 10 min at 72° C.

Then, the gene fragment of VEGF-SEA fusion protein was constructed.

### EXAMPLE 6

### Expression of EGF-SEA and VEGF-SEA Fusion Protein Gene in E. coli

(A) Construction of Expression Plasmid and DNA Sequencing

DNA fragments of fusion protein gene obtained in Example 4 and Example 5 were digested with restriction enzyme SrfI and NotI, pET-34b plasmids were also digested with restriction enzyme SrfI and NotI. The two DNA fragments were ligated into pET-34b with DNA ligase, respectively. DNA ligation reaction was carried out at 16° C for 12 hours. Then, two plasmids containing the fusion protein gene were obtained.

Competent cells of E. coli BL21 were prepared by the calcium chloride method. The two plasmids were introduced into competent E. coli BL21 cells by heat shock. After over-night culture in LB medium containing kanamycin (5 mg/L), single colonies with kanamycin resistance were selected. Plasmids were prepared and purified by common method (T. Maniatis, et al, Molecular cloning, A laboratory manual, Second edition, Cold spring harbor laboratory, 1989), restriction map of plasmids in E. coli was analyzed to confirm that the fusion protein gene had been introduced into E. coli.

In this way, two E. coli strains containing EGF-SEA and VEGF-SEA fusion protein gene respectively were obtained. Strains were conserved at −70° C. in medium containing 15% glycerol.

At last, DNA sequence of EGF-SEA and VEGF-SEA fusion protein gene in the two plasmids was confirmed by DNA sequencing.

SEQ ID NO:1 shown in sequence list is the sequence of epidermal growth factor (EGF)-linker-superantigen(SEA) fusion protein gene: the polypeptide from No 1 to No 53 aa is EGF, the polypeptide from No 54 to No 68 aa is linker, the polypeptide from No 69 to No 301 aa is SEA. Sequence shown in SEQ ID NO:2 is the amino acid sequence of SEQ ID NO:1.

SEQ ID NO:3 shown in sequence list is the sequence of vascular endothelial cell growth factor (VEGF)-linker-superantigen(SEA) fusion protein gene: the polypeptide from No 1 to No 121 aa is VEGF, the polypeptide from No 122 to No 136 aa is linker; the polypeptide from No 137 to No 369 aa is SEA. Sequence shown in SEQ ID NO:4 is the amino acid sequence of SEQ ID NO:3.

(B) Expression of Fusion Protein Gene

E. coli transformants containing the two plasmids respectively were cultured at 37° C. in medium containing kanamycin. Since the two fusion protein genes were under the control of T7 promoter, they were highly expressed in the presence of 1 mM IPTG after further over night incubation.

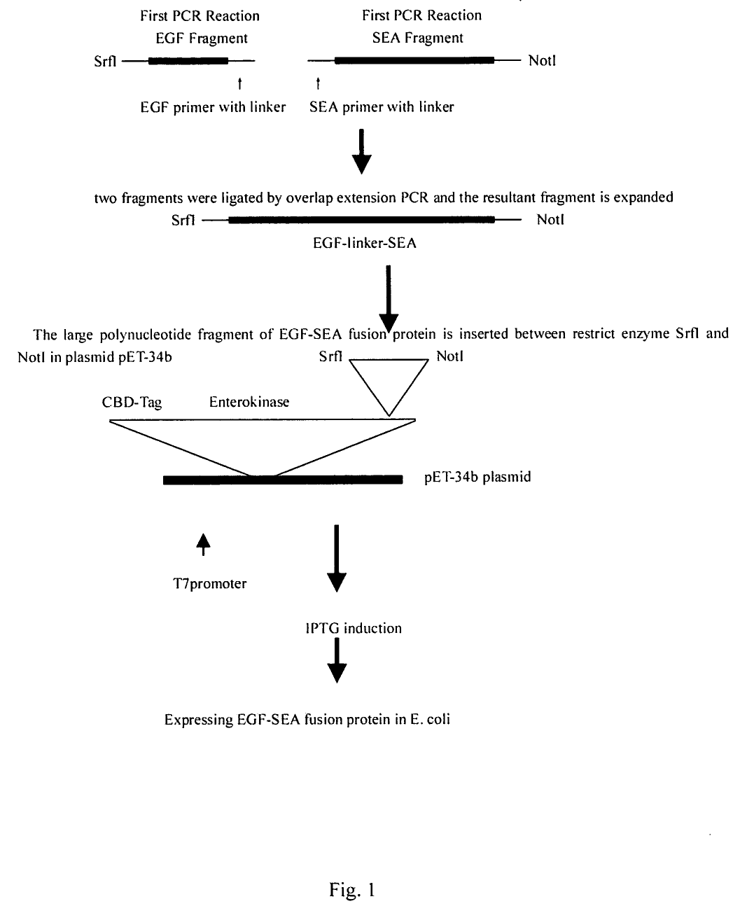

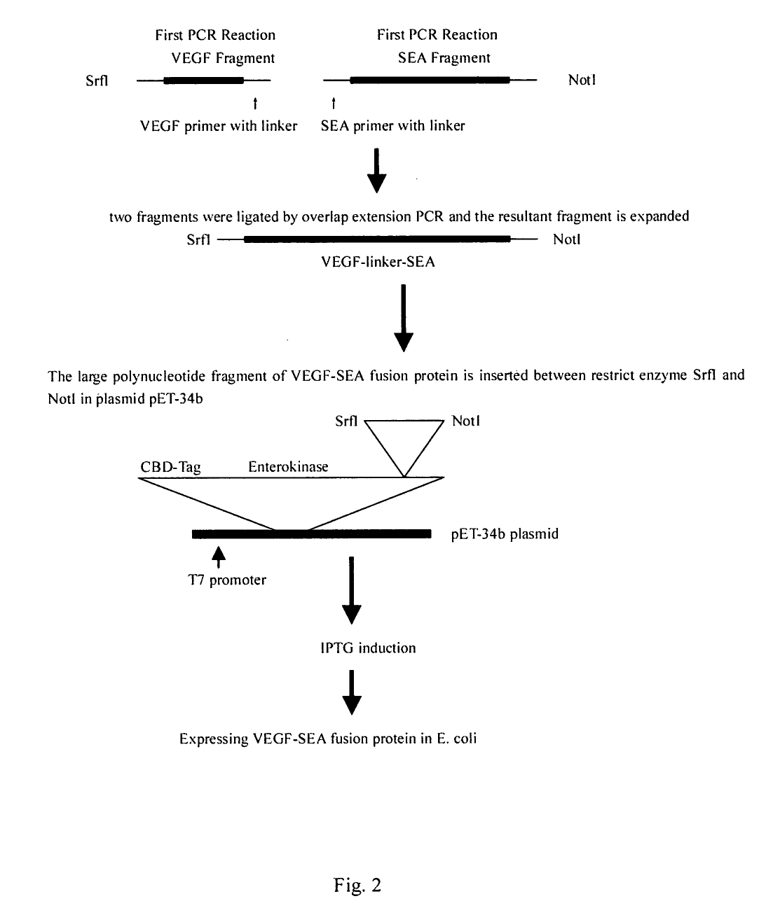

FIG. 1 and FIG. 2 shows the procedures of EGF-SEA and VEGF-SEA fusion protein gene construction and expression, respectively.

### EXAMPLE 7

### Isolation and Purification of EGF-SEA and VEGF-SEA Fusion Protein

The culture medium of two E. coli transformants obtained in Example 6 that highly express EGF-SEA and VEGF-SEA fusion protein respectively was centrifuged at 5000 rpm for 30 min, the cell pellet was collected, washed by 50 mM phosphate buffer (pH 7.0), and then lysed by sonication. The lysate was centrifuged at 10000 rpm for 30 min, the supernatant was collected. Then, the crude extract containing fusion protein was obtained.

pET-34b consists of a CBD fragment, which may serves as a purification Tag. Taking advantage of the CBD fragment, the expressed exogenous protein may be directly purified by using cellulose resin. The method can be commonly used. CBIND ReadyRun Column (Novagen Inc.) was used for purification. The crude extract was applied onto a cellulose column. After the fusion protein containing CBD was adsorbed, the cellulose column was first washed by 20 mM phosphate buffer to eliminate other protein impurities, and then the fusion protein was eluted by phosphate buffer containing 1% cellobiose. The eluted fraction containing fusion protein was collected.

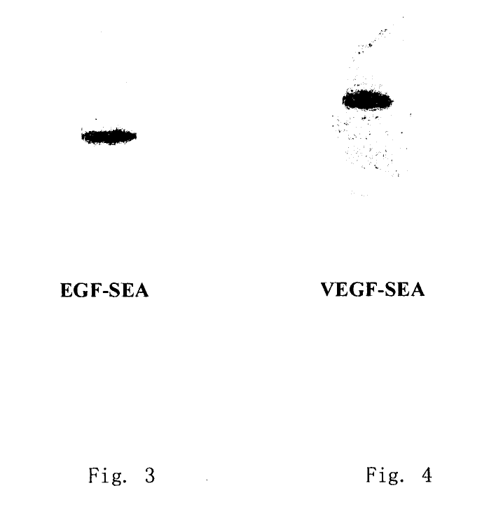

Enterokinase was added to the eluted fraction to cleave CBD fragment. Then the elution fraction was dialyzed against 20 mM phosphate buffer (pH 7.0) at 4° C. to exclude cellobiose. The dialyzed solution was further exposed to cellulose resin. Free CBD fragment was adsorbed by cellulose, while fusion protein without CBD wasn't adsorbed, thus obtaining the purified fusion protein without CBD. FIG. 3 and FIG. 4 show the results of SDS-PAGE of the two fusion proteins. FIG. 3 shows the purified EGF-SEA, while FIG. 4 shows the purified VEGF-SEA.

The N-terminal amino acid sequences of the two proteins were sequenced, they are identical to the N-terminal amino acid sequence of EGF and VEGF respectively.

### EXAMPLE 8

### In vitro Tumor Cell Inhibition Assay of EGF-SEA and VEGF-SEA Fusion Proteins

Normal human peripheral blood progenitor cells (PBMC) and human caucasian larynx carcinoma cells (Hep2) were adjusted to the concentration of approximately 2×10⁴−4×10⁴ cells/ml, the latter carcinoma cells were diluted 5-fold and seeded into 96-well plate, undiluted PBMC cells were added to these plates. Then the effector/target cell ratio between PMBC cell and Hep2 cell was 5:1. Two 96-well plates containing the two cells as described above were prepared. At last, filtration-sterilized EGF-SEA and VEGF-SEA fusion protein were separately added at final concentration of 0.00, 0.05, 0.50, 1.00, 2.00, 3.00, 4.00, 5.00 μg/ml. The 96-well plates were cultured at 37° C. for 48 hours in a CO₂ incubator.

In addition, single PBMC, single EGF-SEA, or single VEGF-SEA fusion protein was separately added to a 96-well plate containing carcinoma cell Hep2 as a control.

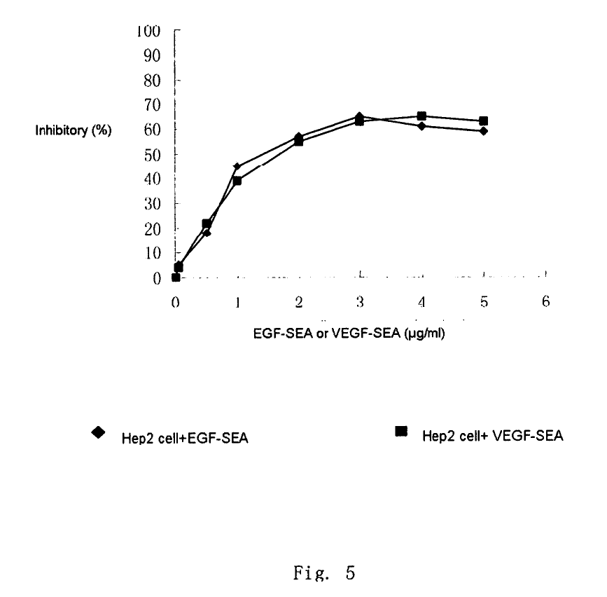

At the effector/target cell ratio of 5:1, EGF-SEA fusion protein showed maximum tumor cell inhibition rate at the concentration of 3 μg/ml. FIG. 5 shows the tumor cell inhibition effect of EGF-SEA and VEGF-SEA fusion protein. The result demonstrates that EGF-SEA and VEGF-SEA fusion protein may activate immune cells.

While in the control experiments that single PBMC, single EGF-SEA or single VEGF-SEA fusion protein were separately added, no obvious tumor cell inhibition was observed.

In the method of the examples hereinabove, the inventor produced different kinds of fusion proteins, wherein the ligand is selected from basic fibroblast growth factor bFGF and FGF family, transforming growth factor-α (TGF-α), interleukin-4, interleukin-2, interleukin-6, interleukin-13, heparin-binding EGF-like growth factor (HB-EGF), insulin-like growth factor (IGF), hepatocyte growth factor (HGF), platelet-derived growth factor (PDGF), nerve growth factor (NGF), placental growth factor (PGF), stem cell factor (SCF), interleukin-8, Ephrin family, Heregulin, erbB ligand, chemokine, angiopoietin (Ang), thrombopoietin (TPO), factor VII, urokinase-type plasminogen activator (uPA), growth hormone releasing hormone (GHRH), gonadotropin-releasing hormone (GRH), α-melanocyte stimulating hormone (α-MSH), gastrin-releasing peptide (GRP), prolactin (PRL), prolactin releasing hormone (PRLH), growth hormone, follicle stimulating hormone (FSH), placental lactogen (PL), chorionic gonadotropin (CG), corticotrophin releasing hormone (CRH), somatostatin (SST), asialoglycoprotein (ASGP), low density lipoprotein (LDL) and transferring (Tf); the superantigen is selected from SEB, SEC, SED, SEE, Streptococcus pyogenes exotoxin, such as SPE-A, SPE-B and SPE-C, and viral protein. These proteins were ligated via a linker to prepare fusion proteins, after expression and purification, these fusion proteins performed encouraging anti-cancer effects in immune cell and cancer cell analysis.

1. A fusion protein, wherein the fusion protein comprises:
a) a ligand that stimulates cancer cell growth and corresponds to receptors overexpressed by cancer cells, or a screened peptide that is affinitive to or antagonist to cancer cell receptors, or a peptide that directly interacts with cancer cell surface;
b) a superantigen that may lead to anti-cancer immune response.

2. A fusion protein according to claim 1, wherein the ligand that stimulates cancer cell growth and corresponds to receptors overexpressed by cancer cells is selected from: epidermal growth factor (EGF) family, vascular endothelial cell growth factor (VEGF) family, basic fibroblast growth factor bFGF and FGF family, transforming growth factor-α (TGF-α), interleukin-4, interleukin-2, interleukin-6, interleukin-13, interleukin-3, granulocyte-macrophage colony-stimulating factor (GM-CSF), heparin-binding EGF-like growth factor (HB-EGF), insulin-like growth factor (IGF), hepatocyte growth factor (HGF), platelet-derived growth factor (PDGF), nerve growth factor (NGF), placental growth factor (PGF), stem cell factor (SCF), interleukin-8, Ephrin family, Heregulin, erbB ligand, chemokine, angiopoietin (Ang), thrombopoietin (TPO), factor VII, urokinase-type plasminogen activator (uPA), growth hormone releasing hormone, gonadotropin-releasing hormone (GRH), α-melanocyte stimulating hormone (α-MSH), gastrin-releasing peptide (GRP), prolactin (PRL), prolactin releasing hormone (PRLH), growth hormone, follicle stimulating hormone (FSH), placental lactogen (PL), chorionic gonadotropin (CG), corticotrophin releasing hormone, somatostatin, asialoglycoprotein, low density lipoprotein and transferring, and other ligands associated with cancers or immune diseases, and their nature variants and artificial variants with more than 70% identity, and artifical polypeptides that interact with cancer cell surface receptors.

3-10. (canceled)

11. A fusion protein according to claim 2, wherein the amino acid sequence of natural variants and artificial variants is at least 70% identical to that of the ligands.

12. A fusion protein according to claim 1, wherein the superantigen that leads to anti-cancer immune response is selected from: Staphylococcal enterotoxin (SE), Streptococcus pyogenes exotoxin (SPE), Staphylococcus aureus toxic shock-syndrome toxin (TSST), Streptococcal mitogenic extotoxin (SME), Streptococcal superantigen (SSA), viral protein and the nature and artificial variants thereof.

13. A fusion protein according to claim 1, wherein the Staphylococcal enterotoxin is selected from SEA, SEB, SEC, SED, SEE, SEG, SHE, SEI, SEJ, SEK, SEL, SEM, SER and SET, wherein the Streptococcus pyogenes exotoxin is selected from SPE-A, SPE-B, SPE-C, SPE-F, SPE-G, SPE-H, SPE-I, SPE-J, SPE-L and SPE-M.

14. A fusion protein according to claim 1, wherein the ligand that stimulates cancer cell growth and corresponds to receptors overexpressed by cancer cells is selected from epidermal growth factor (EGF) and vascular endothelial cell growth factor (VEGF).

15. A fusion protein according to claim 1, wherein the superantigen that leads to anti-cancer immune response is SEA of Staphylococcal enterotoxin family.

16. A fusion protein according to claim 1, wherein the superantigen is SEA protein, and the ligand is selected from epidermal growth factor (EGF) and vascular endothelial cell growth factor (VEGF).

17. A recombinant vector, wherein the vector comprises a nucleotide sequence that encodes the fusion protein according to claim 1.

18. A host cell, wherein the host cell comprises the recombinant vector according to claim 17.

19. A method for producing the fusion protein according to claim 1, wherein the method comprises:
culturing a host cell, the host cell comprises a recombinant vector, the vector comprises a nucleotide sequence that encodes the fusion protein according to claim 1; and
collecting expressed fusion proteins.

20. A method of preparing therapeutic agents for cancer or immune disease treatment comprising: utilizing the fusion protein according to claim 1.
